(19) United States
(12) Patent Application Publication
(10) Pub. No.: US 2005/0282772 A1
(43) Pub. Date: Dec. 22, 2005

(54) New dietary supplement composition for obesity and inflammation

(76) Inventors: Ganga Raju Gokaraju, Andhra Pradesh (IN); Rama Raju Gokaraju, Andhra Pradesh (IN); Venkata Subbaraju Gottumukkala, Andhra Pradesh (IN); Venkateswarlu Somepalli, Andhra Pradesh (IN)

Correspondence Address: RADER FISHMAN & GRAUER PLLC, LION BUILDING, 1233 20TH STREET N.W., SUITE 501, WASHINGTON, DC, 20036, US

(21) Appl. No.: 11/155,486

(22) Filed: Jun. 20, 2005

Related U.S. Application Data
(60) Provisional application No. 60/580,723, filed on Jun. 21, 2004.

(51) Int. Cl. A61K 31/737; A61K 31/7008; A61K 31/19

(52) U.S. Cl. 514/54; 514/574; 514/62; 514/559; 514/419

(57) ABSTRACT

The present invention relates to dietary supplement phytochemical compositions, comprising calcium, potassium double salt of (−)-hydroxycitric acid and glucosamine hydrochloride, and optionally boswellic acids, curcuminoids, 5-hydroxytryptophan, chondroitin sulfate and L-carnitine. The claimed compositions are useful in dietary supplements, nutritional supplements or pharmaceutical preparations for weight loss and inflammatory epidemics.

### FIELD OF THE INVENTION

The present invention relates to dietary supplement phytochemical compositions, comprising calcium, potassium double salt of (−)-hydroxycitric acid and glucosamine hydrochloride, and optionally boswellic acids, curcuminoids, 5-hydroxytryptophan, chondroitin sulfate and L-carnitin. The claimed dietary supplement is useful for weight loss, inflammatory epidemics and as an antioxidant.

### BACKGROUND OF THE INVENTION

Obesity is a complex, multi-factorial and chronic condition characterized by excess body fat resulting from an imbalance between energy expenditure and caloric intake. Low levels of physical activity, sedentary lifestyles, stress, depression and consumption of high-fat and fast foods are responsible for unwanted weight gain. Recent studies have shown that approximately a third of variance in adult body weights result from genetic influences. Leptin, an adipocyte and placenta-derived circulating protein, regulates the magnitude of fat stores in the body leading to obesity. Gastrointestinal peptides, neurotransmitters and adipose tissue may also have an etiologic role in obesity. Obesity and adipose tissue expansion increase the risk of hypertension, type 2 diabetes, arthritis, elevated cholesterol, cancer and serious hormonal imbalances in women, leading to sterility. Low caloric diets with or without exercise can help with temporary weight loss; however, diet and exercise alone have not proven successful for long-term solutions in weight management (H. G. Preuss, et al, Nutrition Research, 2004, 24, 45-48). In addition, supplememtation with drugs that suppress appetite, reduce food intake, increase energy expenditure and effect nutrient partitioning or metabolism have potential efficacy but is unfortunately accompanied by adverse side effects (C. A. Haller and N. L. Benowitz. Adverse cardiovascular and central nervous system events associated with dietary supplements containing ephedra alkaloids. N. Engl. J. Med., 2000, 343, 1833-1838). Herbal and natural products contain Gymnema extract, Garcinia extract, or carnitine are known to prevent fat accumulation through the inhibition of fat absorption, enhancement of fat decomposition, and the enhancement of fat consumption by the body.

Arthritis is a general term describing over 100 different conditions that cause pain, stiffness and inflammation in one or more joints. Obesity has long been associated with greater risk of developing knee arthritis. Arthritis and particularly inflammation is a critical biological process caused by injury, infection, swelling and also occur due to age related factors and this process is known to occur by increased metabolic activity of archidonic acid, which leads into two main pathways, the cyclooxygenase (COX) and lipoxygenase (LOX). Rheumatoid arthritis is a chronic inflammatory condition that affects the lubricating mechanism and cushioning of joints. As a result of this autoimmune disease the bone surfaces are destroyed, which leads to stiffness, swelling, fatigue and crippling pain. Osteoarthritis is the common form of arthritis and results primarily from progressive degeneration of cartilage glycoaminoglycons. The damage is often compounded by a diminished ability to restore and repair joint structures including cartilage. The smooth surface of the cartilage becomes hard and rough creating friction. As a result of this the joint gets deformed, painful and stiff. The major focus for osteoarthritis treatment, should therefore involve agents that not only stimulate the production of biological substances necessary for regeneration of cartilage cells and proper joint function but also diminish pain inflammation. Steroidal and non-steroidal anti-inflammatory drugs are most commonly used remedies for these diseases, but most of the drugs available in the market known to give side effects.

Free radicals play a major role in the progression of a wide range of pathological disturbances and lead to very serious problems like cancer, Alzheimer's, parkinson's, and cardiovascular diseases. In the food industry, free radicals have been found to be responsible in the deterioration of foods during processing and storage. In view of this, considerable attention has been given to the addition of antioxidants in foods and supplementation of antioxidants to biological systems to scavenge free radicals.

Because of proven safety and no side effects of herbal compositions and there is no such composition to address the above problems, it is therefore an object of the present invention to provide a non-toxic supplement, which exhibits weight loss and anti-arthritic properties without deleterious side effects.

It is also an object of the present invention to provide a composition, which in addition to its weight loss effect and anti-inflammatory, is useful as an antioxidant.

### DISCLOSURE OF THE INVENTION

The present invention provides a dietary supplement phytochemical composition comprising (−)-hydroxycitric acid and glucosamine, and optionally boswellic acids, curcuminoids, 5-hydroxytryptophan, chondroitin sulfate and L-carnitin. The claimed dietary supplement is useful as a nutritional supplement, weight loss management, anti-inflammatory and an antioxidant.

Hydroxycitric Acid

The (−)-hydroxycitric acid (HCA) is a naturally occurring acid found in the rinds of the fruit of Garcinia cambogia, Garcinia indica and Garcinia atroviridis. The dried fruit rind of G. cambogia, also known as Malabar tamarind, is commonly used in Southeast Asia (particularly southern India) as a food preservative, flavoring agent and carminative. The primary mechanism of action of (−)-HCA appears to be related to act as a competitive inhibitor of the enzyme ATP-citrate lyase, which catalyzes the conversion of citrate and coenzyme A to oxaloacetate and acetyl coenzyme A (acetyl-CoA). Extensive experimental studies suggest that (−)-HCA suppresses the fatty acid synthesis, lipogenesis and food intake thus leading to weight reduction. In addition to suppression of fatty acid and fat synthesis, (−)-HCA is thought to suppress food intake via loss of appetite by stimulation of liver gluconeogenesis. Various researchers have evaluated HCA for its weight control properties, fat burning properties, lipid level lowering effect, appetite regulation, metabolic rate increase and other effects (K. K. Sakariah, et al. J. Agric. Food Chem., 2002, 50, 10-22 and references cited therein). A number of patents have been granted based on the above studies and various methods of extraction of HCA from the fruit. The isolation and chemical nature of (−)-hydroxycitric acid from Garcinia rind are described in the publication of Lewis, et al, Phytochemistry, 1965, 4, 619-625. Moffett, et al., U.S. Pat. No. 5,656,314 (1997) described a process for the aqueous extraction of (−)-HCA from Garcinia rinds. Ganga Raju, G. et al., U.S. Pat. No. 6,875,891 (2005) described a process for preparing highly water soluble salts of hydroxycitric acid, particularly alkali and alkaline earth metal double salts and its use as dietary supplements and food products to reduce body weight.

Calcium, potassium salt of (−)-HCA (70% free HCA and 0.45% lactone) was used in this formula. The other mono, di- and triple salts of HCA, having (−)-HCA content in the range of 50-75% could also be used for this supplementary composition. The metals selected to make the salts of (−)-HCA are from group IA (Lithium, sodium, potassium, rubidium, cesium, francium) or group IIA (beryllium, magnesium, calcium, strontium, barium) or zinc.

Glucosamine

Glucosamine (an amino saccharide) helps in strengthening the joint structure thereby improving mobility. So far four sources of glucosamine are reported namely glucosamine hydrochloride, glucosamine hydroiodide, glucosamine sulphate and N-acetyl glucosamine. Recent studies have shown the beneficial effects of glucosamine and its relationship with the symptoms of osteoarthritis, the most common form of arthritis (J. B. Houpt, et al., Effect of glucosamine hydrochloride in the treatment of osteoarthritis of the knee. Journal of Rheumatology, 1999, 2423-30). Glucosamine sulfate, which is naturally found in high concentrations in joint structures, is a stable, tasteless and water-soluble nutrient. It is readily absorbed from the intestines, stays in the blood for several hours, and very little is excreted. Glucosamine salts, taken as a dietary supplement, has been shown to exert a protective effect against joint destruction and is selectively used by joint tissues, exerting a dramatic positive effect in reducing arthritic symptoms and promoting healthy joint function. In particular, glucosamine stimulates the body's manufacture of collagen, the protein of the fibrous substance that holds joints together. Collagen is the main component of the shock-absorbing cushion called articular cartilage, and glucosamine is therefore a necessary nutrient in the production of cartilage and synovial fluid. However, the body's production of glucosamine decreases as a person ages, thereby inhibiting the new growth of cartilage destroyed through wear and tear (T. E. Towheed, Current status of glucosamine therapy in osteoarthritis. Arthritis and Rheumatism, 2003, 49, 601-604).

Glucosamine hydrochloride was used in this composition and other forms of glucoasamine, like glucosamine sulfate or N-acetylglucosamine could be used for this composition.

Boswellic Acids

The gum resin of Boswellia serrata (48% boswellic acids) is largely composed of pentacyclic triterpenes called boswellic acids (BAs). BAs have been shown to affect the activity of 5-lipoxygenase and leukocytes, both significantly involved in the inflammatory response. This effect is due to the binding of BAs to 5-lipoxygenase in a way that prevents the binding of its natural substrate, arachidonate (H. P. T. Ammon et al, J. Ethnopharmacol., 1993, 38, 113-119). Additional studies showed that BAs dramatically decreased the migration and total count of leukocytes to arritic.

BAs have also been shown to have a positive effect on arthritic symptoms. In a 1996 study in rats, BAs prevented inflammation and arthritic activity in developing and established arthritis. Anti-inflammatory agents often decrease the content and synthesis of glycosaminoglycans, important components of cartilage degraded in arthritis. BAs reduce the synthesis, but not the total content of glycosaminoglycans, suggesting that BAs inhibit the destruction of cartilage. In support of this possibility, it has been found that BAs inhibit several cartilage-destructive enzymes, including leukocyte elastase and beta-glucoronidase. Pentacyclic triterpenes from sources other than Boswellic acids inhibit 5-lipoxygenase but do not additionally inhibit cartilage-destructive enzymes.

BAs are well tolerated. In acute, sub-acute and chronic toxicity studies with mice, rats and monkeys, no adverse effects or deaths were recorded. Even at doses of 1000 mg/kg, BAs had no ulcerogenic effects, as does aspirin (H. P. T. Ammon et al, Eur. J. Med. Res., 1997, 2, 37-43). Boswellic acids (48% by HPLC and 85% by gravimetric) was used in this study. Boswellic acids in different concentrations could also be used for this phytochemical composition.

Curcuminoids

Curcuminoids, are characteristic yellow coloured constituents of turmeric (Curcuma longa) and are widely using in foods and cosmetics. These compounds were reported to posses antioxidant, anti-inflammatory, anticancer and antiviral properties. Studies show curcuminoids have a potent effect on the inflammatory response. These directly block the activity of COX-2 and 5-LOX in rat and human cell lines. Curcuminoids also inhibits the synthesis of new COX-2 protein and mRNA. The potency of curcuminoids has been evaluated in comparison to other anti-inflammatory drugs. The activity of curcuminoids was compared to phenylbutazone in patients with rhenumatoid arthritis and gave significant improvement in the duration of morning stiffness, walking time and joint swelling compared to phenylbutazone. Further, curcuminoids have a lower ulcerogan index (0.6) than a similar active dose of phenylbutazone (1.7) based on an animal study. Curcuminoids, used in this composition was extracted from curcuma longa (turmeric), having total curcuminoids (curcuminoid, demethoxycurcuminoid and bisdemethoxycurcuminoid) concentration is in the range of 90-95% by HPLC.

5-Hydroxytryptophan

5-Hydroxytryptophan (5-HTP) is a natural compound isolated from the seeds of an African plant called Griffonia simplicifolia. Substances which increase brain serotonin (5-hydroxytryptamine, 5-HT) are effective anorectic agents to help obese patients lose weight and to decrease cravings for sweets and carbohydrates. Experimental studies proved that 5-HTP increases brain serotonin (5-HT) and its LD₅₀ value is negligible even at the dose of 2500 mg/kg, so it is a safe product. 5-Hydroxytryptophan (95%) was used in this composition. Other concentrations of 5-HTP could also be used.

Chondroitin

Chondroitin is high viscosity mucopolysaccharides with N-acetylchondrosine as a repeating unit and with one sulfate group per saccharide unit. These biological polymers act as the flexible connecting matrix between the tough protein filaments in cartilage to form a polymeric system similar to reinforced rubber. Chondroitin 4-sulfate and chondroitin 6-sulfate are the most abundant mucopolysaccaharides in the body and occur both in skeletal and soft connective tissue, which inhibits cartilage degradation.

L-(−)-Carnitine

L-(−)-Carnitine is a dipeptide manufactured in the liver from the amino acids lysine, methionine, and vitamins B6, C, B3, and the mineral iron. It plays an important part in energy metabolism, in the breakdown of fatty acids, in the eukaryotic organism. It is a vitamin-like substance, which has been admixed to food products or administered as dietary supplement or pharmaceutical dosage form. It has been used in the cardiovascular field in the treatment of acute and chronic myocardial ischaemia, angina pectoris, cardiac arrhythmias and insufficiency, and peripheral vascular diseases. The dietary reference intake is 1000-2000 mg per day.

The present invention provides a new phytochemical composition by mixing appropriate unit doses of the above phytochemicals to get the composition suitable for reduing obesity, arthritis and other free radical mediated diseases.

Active new phytochemical compositions of the present invention may be produced by the procedures described herein or variations thereof, which will be apparent to those skilled in the art.

A further aspect of the present invention is a pharmaceutical formulation comprising a composition as described above in a pharmaceutically acceptable carrier (e.g., an aqueous or a non aqueous carrier).

A still further aspect of the present invention is a method of treating ‘obesity, inflammatory diseases, free radical mediated diseases’ comprising administering to a human or animal subject in need thereof a treatment effective amount (e.g., an amount effective to treat, slow the progression of, etc.) of a composition as described above.

Preferred embodiments relating to the different compositions of the subject invention are illustrated in the examples given below:

### EXAMPLE 1

A phytochemical composition was prepared by mixing unit doses of the following components: calcium, potassium double salt of (−)-hydroxycitric acid (4 g) and glucosamine hydrochloride (1.5 g).

The analytical characteristics of the mixture thus obtained are, (−)-HCA is 50.6%; lactone is 0.4%; calcium is 7.9%; potassium is 13.2% and glucosamine (as free base) is 23.0%.

### EXAMPLE 2

A phytochemical composition was prepared by mixing unit doses of the following components: calcium, potassium double salt of (−)-hydroxycitric acid (3.5 g) and glucosamine hydrochloride (2 g).

The analytical characteristics of the mixture thus obtained are, (−)-HCA is 45.1%; lactone is 0.3%; calcium is 7.1%; potassium is 11.9% and glucosamine (as free base) is 29%.

### EXAMPLE 3

A phytochemical composition was prepared by mixing unit doses of the following components: calcium, potassium double salt of (−)-hydroxycitric acid (4 g), glucosamine hydrochloride (1.5 g) and boswellic acids (48%; 300 mg).

The analytical characteristics of the mixture thus obtained are, (−)-HCA is 48.5%; lactone is 0.5%; calcium is 7.4%; potassium is 11.8%; glucosamine (as free base) is 22.1 % and total boswellic acids is 2.2%.

### EXAMPLE 4

A phytochemical composition was prepared by mixing unit doses of the following components: calcium, potassium double salt of (−)-hydroxycitric acid (3.5 g), glucosamine hydrochloride (2 g) and boswellic acids (48%; 300 mg).

The analytical characteristics of the mixture thus obtained are, (−)-HCA is 43.3%; lactone is 0.4%; calcium is 6.9%; potassium is 10.8%; glucosamine (as free base) is 29.1% and total boswellic acids is 2.5%.

### EXAMPLE 5

A phytochemical composition was prepared by mixing unit doses of the following components: calcium, potassium double salt of (−)-hydroxycitric acid (3.5 g), glucosamine hydrochloride (2 g), boswellic acids (48%; 300 mg) and 5-hydroxytryptophan (95%, 200 mg).

The analytical characteristics of the mixture thus obtained are, (−)-HCA is 40.7%; lactone is 0.3%; calcium is 6.5%; potassium is 9%; glucosamine (as free base) is 27%; total boswellic acids is 2.4% and 5-HTP is 3.2%.

### EXAMPLE 6

A phytochemical composition was prepared by mixing unit doses of the following components: calcium, potassium double salt of (−)-hydroxycitric acid (4 g), glucosamine hydrochloride (1.5 g), boswellic acids (48%; 300 mg) and curcuminoids (95%; 200 mg).

The analytical characteristics of the mixture thus obtained are, (−)-HCA is 45.3%; lactone is 0.4%; calcium is 7.4%; potassium is 11.6%; glucosamine (as free base) is 19.9%; total boswellic acids is 2.3% and total curcuminoids is 3.3%.

### EXAMPLE 7

A phytochemical composition was prepared by mixing unit doses of the following components: calcium, potassium double salt of (−)-hydroxycitric acid (3.5 g), glucosamine hydrochloride (2 g), boswellic acids (48%; 300 mg) and curcuminoids (95%; 200 mg).

The analytical characteristics of the mixture thus obtained are, (−)-HCA is 40.5%; lactone is 0.4%; calcium is 7.3%; potassium is 11.8%; glucosamine (as free base) is 28%; total boswellic acids is 2.3% and total curcuminoids is 3.8%.

### EXAMPLE 8

A phytochemical composition was prepared by mixing unit doses of the following components: calcium, potassium double salt of (−)-hydroxycitric acid (3.5 g), glucosamine hydrochloride (2 g), boswellic acids (48%; 300 mg), 5-hydroxytryptophan (95%, 200 mg) and curcuminoids (95%; 200 mg).

The analytical characteristics of the mixture thus obtained are, (−)-HCA is 39.1%; lactone is 0.3%; calcium is 6.3%; potassium is 8.8%; glucosamine (as free base) is 26.8%; total boswellic acids is 2.3%; 5-HTP is 3.1% and total curcuminoids is 3.1%.

### EXAMPLE 9

A phytochemical composition was prepared by mixing unit doses of the following components: calcium, potassium double salt of (−)-hydroxycitric acid (3.5 g), glucosamine hydrochloride (2 g), boswellic acids (48%; 300 mg), 5-hydroxytryptophan (95%, 200 mg), curcuminoids (95%; 200 mg), chondroitin sulfate (200 mg) and L-carnitin (200 mg).

The analytical characteristics of the mixture thus obtained are, (−)-HCA is 37%; lactone is 0.3%; calcium is 6%; potassium is 8.2%; glucosamine (as free base) is 25%; total boswellic acids is 2.2%; 5-HTP is 2.9%; total curcuminoids is 2.9% and L-carnitine is 3%.

5-Lipoxygenase Activity: The new phytochemical compositions were screened for their 5-Lipoxygenase inhibitory potential using colorimetric method. The assay mixture contained 50 mM phosphate buffer pH 6.3, 5-Lipoxygenase, various concentrations of test substances in dimethyl sulphoxide and linoleic acid in a total volume of 0.5 mL, after 5 min incubation of above reaction mixture, 0.5 mL ferric xylenol orange reagent was added and OD was measured after two minutes at 585 nm using spectrophotometer. Controls were run along with test in a similar manner except using vehicle instead of test substance solution. Percent inhibition was calculated by comparing absorbance of test solution with that of control.

Superoxide Free Radical-Scavenging Activity. The superoxide free radical-scavenging activity was determined by the NBT (nitro blue tetrazolium) method. The reaction mixture contained EDTA (6.6 mM), NaCN (3 μg), riboflavin (2 μM), NBT (50 μM), various concentrations of the test drug in ethanol and a phosphate buffer (58 mM, pH 7.8) in a final volume of 3 ml. Optical density was measured at 560 nm. The test tubes were uniformly illuminated with an incandescent lamp for 15 min, after which the optical density was measured again at 560 nm. The percentage inhibition and superoxide radical generation was measured by comparing the absorbance values of the control and those of the test compounds.

DPPH Free Radical Scavenging Activity. DPPH (1,1-diphenyl-2-picryl-hydrazyl) radical scavenging activity was measured based on the reduction of methanolic solution of the colored DPPH. Free radical scavenging ability of the test drug in ethanol added to the methanolic solution of DPPH is inversely proportional to the difference in initial and final absorption of DPPH solution at 516 nm. The reaction mixture contained 1×10⁻⁴ mM methanolic solution of DPPH and various concentrations of test drugs. The percentage inhibition was determined by comparing the absorbance values of test and control tubes.

The new phytochemical compositions of this invention are found to show 5-lipoxygenase activity (Table 1; 5-lipoxygenase activity is expressed in % of inhibition at 100 μg concentrations; higher the % inhibitory values, higher is the activity).

The new phytochemical compositions of this invention are found to show antioxidant activity (Table 1; antioxidant activity is measured by two different mechanisms (a) Nitro blue tetrazolium (NBT) (b) DPPH free radical scavenging methods and are expressed in % of inhibition at 500 μg concentrations; higher the % inhibitory values, higher is the activity).

TABLE 1
Anti-inflammatory* and antioxidant activity** of phytochemical compositions

| | | % inhibition of 5-LOX | % inhibition of NBT free | % inhibition of DPPH free |
|---|---|---|---|---|
| | | activity at 100 μg | radical scavenging activity | radical scavenging activity |
| S. No. | Composition | concentrations | at 500 μg concentrations | at 500 μg concentrations |
| 1 | HCA (70%) | 0.2 | 25.5 | 0 |
| 2 | Glucosamine HCl | 0 | 5.6 | 2.8 |
| 3 | Composition 1 | 1.5 | 32.1 | 2.6 |
| | (HCA = 4 g, | | | |
| | glucosamine = 1.5 g) | | | |
| 4 | Composition 2 | 1.7 | 35.9 | 3.3 |
| | (HCA = 3.5 g, | | | |
| | glucosamine = 2 g) | | | |
| 5 | Composition 3 | 2.3 | 45.1 | 14.6 |
| | (HCA = 4 g, | | | |
| | glucosamine = 1.5 g, | | | |
| | boswellic acids = 300 | | | |
| | mg) | | | |
| 6 | Composition 4 | 2.4 | 47.1 | 15.1 |
| | (HCA = 3.5 g, | | | |
| | glucosamine = 2 g, | | | |
| | boswellic acids = 300 | | | |
| | mg) | | | |
| 7 | Composition 5 | 4.8 | 64.9 | 86.1 |
| | (HCA = 4 g, | | | |
| | glucosamine = 1.5 g, | | | |
| | boswellic acids = 300 | | | |
| | mg, curcuminoids = 200 | | | |
| | mg) | | | |
| 8 | Composition 6 | 3.8 | 66.6 | 85.8 |
| | (HCA = 3.5 g, | | | |
| | glucosamine = 2 g, | | | |
| | boswellic acids = 300 | | | |
| | mg, curcuminoids = 200 | | | |
| | mg) | | | |

*Anti-inflammatory activity was determined by 5-lipoxygenase method.
**Antioxidant efficacy was determined by superoxide free radical (NBT) and DPPH free radical scavenging methods.

1. A dietary supplement phytochemical composition for weight loss and inflammatory diseases comprising (−)-hydroxycitric acid salt and glucosamine salt and optionally boswellic acids, curcuminoids, 5-hydroxytryptophan, chondroitin sulfate and L-carnitin.

2. The dietary supplement phytochemical composition as claimed in claim 1, wherein (−)-hydroxycitric acid salt is selected from mono or di or tri salts of (−)-hydroxycitric acid.

3. The dietary supplement phytochemical composition as claimed in claim 2, wherein said (−)-hydroxycitric acid salt has hydroxycitric acid content between 40-90% and lactone content between 0-50%.

4. The dietary supplement phytochemical composition as claimed in claim 2, wherein the (−)-hydroxycitric acid salt is selected from group IA and/or group IIA and/or zinc salts of (−)-hydroxycitric acid and preferably calcium and potassium double salt of (−)-hydroxycitric acid or calcium, potassium and magnesium triple salt of (−)-hydroxycitric acid or mixtures thereof.

5. The dietary supplement phytochemical composition as claimed in claim 1, wherein the glucosamine salt is selected from glucosamine hydrochloride or glucosamine sulfate or N-acetyl glucosamine.

6. The dietary supplement phytochemical composition as claimed in claim 1, wherein the boswellic acids is an enriched extract from Boswellia serrata gum resin, said extract having between 10-100% of boswellic acids.

7. The dietary supplement phytochemical composition as claimed in claim 1, wherein the curcuminoids is an enriched extract from Curcuma longa, said extract having between 90-95% of the curcuminoids .

8. The dietary supplement phytochemical composition as claimed in claim 1, comprising 40-80% of calcium and potassium double salt of (−)-hydroxycitric acid and 20-50% of glucosamine hydrochloride.

9. The dietary supplement phytochemical composition as claimed in claim 1, comprising 40-80% of calcium and potassium double salt of (−)-hydroxycitric acid, 20-50% of glucosamine hydrochloride and 2.5-10% of boswellic acids.

10. The dietary supplement phytochemical composition as claimed in claim 1, comprising 40-80% of calcium and potassium double salt of (−)-hydroxycitric acid, 20-50% of glucosamine hydrochloride, 2.5-10% of boswellic acids and 2.5-10% of curcuminoids.

11. The dietary supplement phytochemical composition as claimed in claim 1, is in the form of a therapeutic formulation, for treating obesity.

12. The dietary supplement phytochemical composition as claimed in claim 1, is in the form of a therapeutic formulation, for treating arthritis and inflammatory diseases.

13. The dietary supplement phytochemical composition as claimed in claim 1 is in the form of a therapeutic formulation, for treating free radical mediated cardiovascular diseases.

14. The dietary supplement phytochemical composition as claimed in claim 1, is in the form of a dietary or nutraceutical formulation.

15. The dietary supplement phytochemical composition as claimed in claim 1, is in the form of a beverage.

16. The dietary supplement phytochemical composition as claimed in claim 1 is in the form of a cosmetic formulation.

17. A pharmaceutical formulation comprising a composition according to claim 1 and pharmaceutical acceptable carrier.

18. The pharmaceutical formulation according to claim 16, wherein said carrier is an aqueous or non-aqueous carrier.
